(19) United States
(12) Patent Application Publication
(10) Pub. No.: US 2025/0221637 A1
(43) Pub. Date: Jul. 10, 2025

(54) COIN-SIZED, FULLY INTEGRATED AND MINIMALLY INVASIVE CONTINUOUS GLUCOSE MONITORING SYSTEM (CGMs) BASED ON ORGANIC ELECTROCHEMICAL TRANSISTORS

(71) Applicant: The University of Hong Kong, Hong Kong (HK)

(72) Inventors: Shiming ZHANG, Hong Kong (HK); Jing BAI, Hong Kong (HK); Xinyu TIAN, Hong Kong (HK); Dingyao LIU, Hong Kong (HK)

(21) Appl. No.: 18/818,762

(22) Filed: Aug. 29, 2024

Related U.S. Application Data
(60) Provisional application No. 63/617,436, filed on Jan. 4, 2024.

(51) Int. Cl. A61B 5/145; A61L 31/14; A61M 37/00

(52) U.S. Cl. CPC A61B 5/14532; A61L 31/145; A61M 37/0015

(57) ABSTRACT

This invention provides a coin-sized, fully integrated and wearable continuous glucose monitoring system (CGMs) via combining cutting-edge technologies from the intersecting fields of biosensors, minimally invasive tools, and hydrogels. The invention includes three major parts: 1) an emerging biochemical amplifier, the organic electrochemical transistor (OECT), to improve sensitivity beyond traditional electrochemical modules; 2) a microneedle array for interstitial-fluid (ISF) sampling with reduced pain during skin penetration; and 3) a tough, adhesive enzymatic-hydrogel-membrane to enhance reliability of glucose sensing on skin. Unlike conventional CGMs, the employed OECT amplifier empowers the CGM (OECT-CGMs) with a high anti-noise ability, an on-demand-tunable sensitivity and current regeneration ability, enabling long-term stable glucose sensing within specific clinical ranges (1˜20 mM). This work paves the way for the development of next-generation CGMs that can simultaneously deliver high and adjustable sensitivity, minimal invasiveness, and improved wearability.

### CROSS-REFERENCE TO RELEVANT APPLICATIONS

The present application claims priority from a U.S. provisional patent application Ser. No. 63/617,436 filed Jan. 4, 2024, and the disclosure of which are incorporated by reference in their entirety.

### FIELD OF THE INVENTION

The present invention generally relates to the field of wearable continuous glucose monitoring systems. More specifically the present invention relates to an organic electrochemical transistor-based continuous glucose monitoring system, equipped with lower limit of detection, high resolution, high anti-noise ability, auto-calibration and current regeneration abilities.

### BACKGROUND OF THE INVENTION

Diabetes mellitus is one of the most malignant chronic diseases threatening human health. Improper interventions for diabetes mellitus patients may cause sudden hypoglycemia and increase the risk of complications, emphasizing the importance of accurate detection of blood glucose levels. The last two decades have witnessed intensive progress in continuous glucose monitoring system (CGMS). CGMS can inform, notify, and alert patients with diabetes of sustained hyperglycemia and incident hypoglycemia and provide necessary information to closed-loop blood glucose control systems. Wearable CGM systems have been regarded as a promising candidate for offering personalized lifestyle routine suggestions through round-the-clock continuous blood glucose monitoring. However, current wearable CGM devices still suffer from problems like pain while implanting the sensor and maintaining high accuracy while continuously tracking blood glucose levels. Therefore, the development of next-generation CGMs with higher sensitivity, minimally invasiveness and comfort wearability has become a major research focus of the community to promote diabetes management in different scenarios.

Organic electrochemical transistors (OECTs) have recently emerged as one of the most promising technologies as next-generation biosensors due to the combination of the merits of electrochemical transducers and transistor amplifiers. OECTs based on ionic-electronic mixed semiconductors, e.g., poly(3,4-ethylenedioxythiophene): poly (styrene-sulfonate) (PEDOT:PSS), can operate in aqueous environments at low voltage (<1 V) with low-power consumption while maintaining stable performance over months. The operation of OECTs relies on the electrochemical doping/de-doping process of the ionic-electronic mixed semiconducting channel in contact with the electrolyte, where mobile ions can penetrate in between and then modulates the whole channel bulk. These properties endow OECTs-based biosensors with the ability to amplify weak electrochemical signals in situ with high signal-to-noise (SNR) ratio at low voltage and low power. As a result, OECT technology can effectively address the low-power/high-SNR tradeoff intrinsic to biomedical sensors.

To date, research on OECTs-based glucose biosensors is mostly concentrated on enhancing sensors' performance metrics like amplification ability (i.e., transconductance), mechanical stretchability, and operational stability. The following has been reported in various reports and academic articles: an OECT-based glucose sensor working in a neutral pH environment; a highly selective glucose sensor by modifying the gate electrode of the OECTs with carbon nanotubes (CNTs; an OECTs-based glucose sensor with micromolar sensitivity where the gate electrode is coated with a gel containing glucose oxidase (GOx); stretchable OECTs for glucose detection, where the device remains functional under up to 30% of strain. A most recent paper reported a high-transconductance OECT demonstrating sensitive detection of glucose with a transconductance of 180 mS, achieved using the interdigitated-electrode structure.

While OECT glucose biosensors have been extensively researched, their practical application in a wearable CGM scenario has not been explored due to a lack of system-level development strategies. To maximize their competitiveness as next-generation CGMs, OECT-glucose sensors need to be integrated with miniaturized readout systems for comfort wearability, and minimally invasive sampling technologies to reduce pain during skin penetration and minimize the induced risk during the wearing period. Furthermore, strategies that ensure skin-interfacing stability and reliability during long-term use are also an indispensable part of filling in the missing pieces of the system puzzle.

In this invention, an intrinsically stretchable glucose-sensing enzyme membrane is developed by combining: i) a mixed-conducting polymer hydrogel and ii) a stretchable redox hydrogel which is enzyme-loaded via a covalent bonding approach. Specifically, a 3D interpenetrated structure is provided, in which the strategic use of a PEDOT:PSS conducting network reduces the distance between the enzymes and the electronically conducting networks (i.e., electrodes). This approach promotes both direct electron transfer (DET) and mediated electron transfer (MET), representing a significant advancement in enzyme technologies for stretchable molecular biosensing.

This invention has the potential to impact the growth of a new research direction, stretchable enzyme technology. This technology, customized for emerging wearable and tissue-compatible biosensing applications, will significantly improve the strain robustness of membranes, electrodes, and devices, thus promoting their wider deployment at soft biological interfaces. For example, it can serve as a potential supplement to the current three major glucose oxide enzyme technologies to be used for developing skin-conformable CGMs. These sensors can be easily incorporated into various soft medical wearables for translational applications, such as sweat-sensing patches and wound-monitoring bandages. Besides, a seamless integration of these membranes with cells, tissues, and organs will open new possibilities to study or mimic the biochemical processes in the soft biological world. The present invention addresses this need.

### SUMMARY OF THE INVENTION

In connection to the above challenges and issues, this invention provides herewith a wearable prototype of OECT-CGM system for diabetes healthcare management.

In an aspect of the present invention, the organic electrochemical transistor-based continuous glucose monitoring system is provided with minimal intrusiveness and no blood contact, comprising a hollow microneedle patch, an adhesive and stretchable enzymatic hydrogel sensing membrane, an organic electrochemical transistor-based glucose sensor, and a 3D printed resin encapsulation case coated with evaporated metal. Specifically, the organic electrochemical transistor is capable of current regeneration, sensitivity adjustment and self-calibration.

In an embodiment, the adhesive and stretchable enzymatic hydrogel sensing membrane comprises an interpenetrating polymer network hydrogel comprising a sodium alginate first network and a polyacrylamide second network, a semipermeable bioadhesive elastomer, and glucose oxidase.

In other embodiment, the evaporated metal coating on the 3D printed resin encapsulation case is selected from gold (Au) or platinum (Pt).

In another embodiment, the organic electrochemical transistor-based continuous glucose monitoring system is capable of tracking cell glucose metabolism and clinical blood glucose concentrations within a glucose concentration range of 10⁻⁶ M to 10⁻¹ M.

In yet another embodiment, the organic electrochemical transistor-based continuous glucose monitoring system of the present invention has a dimension of less than 2 cm×2 cm×0.5 cm.

In yet other embodiment, the organic electrochemical transistor-based continuous glucose monitoring system achieves a high signal-to-noise ratio of at least 50 dB.

In a further embodiment, the current regeneration of the organic electrochemical transistor of the system comprises adjusting the gate voltage of the organic electrochemical transistor.

In another further embodiment, the sensitivity adjustment of the organic electrochemical transistor of the system comprises controlling the transconductance to adjust the anti-noise ability; and adjusting the gate voltage to adjust the linear range of detection.

In other further embodiment, the self-calibration of the organic electrochemical transistor of the system comprises normalizing the transconductance curves.

### BRIEF DESCRIPTIONS OF DRAWINGS

FIG. 1 illustrates the concept and design principle of the OECT-CGM system. The system consists of: (i) a microneedle patch for bridging the ISF and the OECT sensor, (ii) a tough and adhesive enzymatic hydrogel sensing membrane, (iii) an OECT glucose sensor, (iv) a miniaturized readout system for real-time communication with peripheral devices, such as a cell phone, and (v) a 3D printed resin encapsulation case. Stacking integration strategy is employed to achieve a size of 6.5 mm in height and 14 mm in radius.

FIGS. 2A to 2I provides an overview to the fabrication and characterization of OECT-based glucose sensor. FIG. 2A is a schematic diagram of the OECT glucose sensor and the sensing mechanism. FIG. 2B shows the optical image of a OECT glucose sensor fabricated on a PI substrate. FIG. 2C show output curves of flexible OECTs, where V_{ds} are swept from 0 V to −0.8 V with a step of 20 mV. FIG. 2D are transfer curves of the OECT with V_{g} swept from −0.1 V to 0.8 V, with a step of 20 mV, repeating for 40 cycles, indicating the stability of OECT for biosensing. FIG. 2E is the real-time current response (ΔI_{ds}) of OECT-based glucose sensor in response to glucose concentration changing from 10⁻⁶ mM to 10⁻¹ mM. FIG. 2F shows the response to potential interfering substances, including ascorbic acid and urea of the OECT glucose sensor, indicating the selectivity of the sensor. FIG. 2G shows the real-time current response (ΔIds) of OECT-based glucose sensor in response to glucose concentration changing from 10⁻⁴ mM to 10⁻² mM under different levels of environmental noise. FIG. 2H shows the real-time current response (ΔI, current of the working electrode) of an electrochemical-based glucose sensor in response to glucose concentration changing from 10⁻⁶ mM to 10⁻¹ mM under different levels of environmental noise. Three white noises with different amplitudes (10.0 μA, 1.0 μA and 0.2 μA) are generated by the function generator. The three different noises are superimposed on the original OECT-based glucose sensor current response curve and the electrochemical glucose sensor current response curve respectively in the data processing stage. Even if the amplitude of the applied noise reaches 10.0 μA, the current response curve of the OEC-based sensor is still clearly distinguishable under different glucose concentrations. But when the amplitude of the applied noise reaches 1.0 μA, the current response curve of the electrochemical sensor is already submerged by the noise. This proves that the OECT-based sensor has a good ability to resist transmission noise. FIG. 2I shows the SNR of OECT glucose sensor and electrochemical-based glucose sensor under application of artificial noise (n=10). Data are presented as means±SD.

FIGS. 3A to 3H summarizes the integrated OECT glucose sensing platform with tunable sensitivity, current regeneration ability, and self-calibration ability. FIG. 3A shows optical images of the PERFECT readout system customized for wearable OECT characterizations. FIG. 3B is a circuit diagram and main components of the PERFECT system.

FIG. 3C shows a comparison of the glucose response (2 mM to 14 mM, with a step of 2 mM) of the OECT sensor measured by PERFECT and the lab-used SMU. FIG. 3D shows the response of OECT glucose sensors to different glucose levels (2 mM to 18 mM) at different Vg. FIG. 3E shows the linear fitting of the results in FIG. 3D, which demonstrates the tunable sensitivity by controlling the V_{g} 0.2 V and 0.3 V, respectively) of OECTs. FIG. 3F shows the current response of OECT glucose sensors upon increasing the glucose concentration to 10 mM (before calibration). FIG. 3G shows the calibrated current response of OECT glucose sensors upon increasing the concentration to 10 mM (after calibration). FIG. 3H shows the calibration process of the OECT glucose sensors performed with their specific transfer curves and the associated gₘ.

FIGS. 4A to 4H provides an overview to the prototyping and in vivo test of a fully-integrated and minimally-invasive wearable OECT-CGM system. FIG. 4A shows the optical image of wearable OECT-CGM on the skin and a 3D-printed microneedle array (left); the glucose diffusion mechanics between ISF via microneedle and IPN hydrogel (middle); and the scanning electron microscope (SEM) image of the hollow microneedle arrays (side and top views) (right), respectively. FIG. 4B illustrates adhesive and tough double-network IPN hydrogel composed of PAAm hydrogel and sodium-alginate hydrogel (left) and optical images of the peeling-off process of the adhesive and tough hydrogel (right). FIG. 4C shows the time lag comparison of the OECT-sensor with/without hydrogel filler. FIG. 4D shows a comparison of size of the OECT-CGM with Dexcom glucose sensor (G6). FIG. 4E shows a comparison of real-time sensing results of the wearable OECT-CGM system with Dexcom G6. FIG. 4F shows the MARD of OECT-CGM (calculated with 12 sampling points within 1000 seconds). FIG. 4G shows the in vivo test setup image of the OECT-CGM for tracking the blood glucose level with a rat model. FIG. 4H shows the in vivo glucose measurement results obtained using the OECT-CGM and a glucometer (black, 16 data points of the glucose concentration collected by a glucometer; red, relative channel current change of the OECT-based glucose sensor measured by the OECT-CGM). a.u. arbitrary units.

FIG. 5. Shows an exploded view of the OECT-CGM system. The OECT-CGM system is assembled by the systematic integration of four main components: i) a miniaturized personalized electronic reader for electrochemical transistors (PERFECT), ii) an OECTs-based glucose sensor to convert changes of glucose concentration to the change in current flowing in a semiconducting channel, I_{ds}, ii) a hollow microneedle array for interstitial fluid (ISF) glucose collection with the length of 0.5 mm to achieve minimal invasions, iv) an enzymatic, tough and adhesive hydrogel layer, with a thickness of 0.15 mm, to improve adhesion and robustness under deformation, effectively bridging the ISF and the sensor for accurate and stable glucose monitoring. v) The replaceable 2036 is used for the independent power supply of the whole system. vi) 3D-printed resin enclosures to encapsulate the whole system.

FIG. 6 shows the current response of the OECT glucose sensor. The sensor is first soaked in PBS solution. After adding 2 mL of 10 mM and 20 mM glucose solution to the test container at Time=46 s and Time=88 s, respectively, the current in the channel of the OECT glucose sensor decreased to 697 μA and 680 μA, with V_{g}=0.2 V.

FIG. 7 demonstrates the signal amplification of the OECT glucose sensor. The power amplification of biosignals in the OECT primarily arises from the integration of gate-to-channel current (I_{g}) by the channel. The product of gate voltage (V_{g}) and gate current (I_{g}) represents the input power of the signal. The product of channel voltage (V_{ds}) and channel current change (ΔI_{ds}) represents the output power. The OECT glucose sensor demonstrated a power amplification of 19.66 dB. n=10. Data are means±SD.

FIG. 8 shows optical images of the prototype of microneedle patch with wearable PERFECT readout system. The overall dimensions of the device are modest, measuring 15 mm in both length and width, with a thickness of 2.5 mm. This compact size allows for easy portability and discreet usage. The inclusion of microneedles in our design aims to provide users with a minimally invasive experience while ensuring accurate glucose level measurements.

FIG. 9 shows the optical image of the prototype of the microneedle array with metalized surface. The microneedle array boasts a 10×10 configuration with sharpened tapered microneedles set at a 15-degree angle for optimal skin penetration. Sized at 10 mm×10 mm×2.2 mm, it aligns seamlessly with the CGM readout system. The fabrication process begins with precision 3D printing to achieve the desired geometry. Next step involves magnetron sputtering, metallizing the surface with a layer of gold (30 nm). This metallization enhances structural strength, ensuring durability, and facilitates smoother application.

FIG. 10A demonstrates the colorimetric test of diffusion in the double-network hydrogel layer. Prior to gelation, the dual-network hydrogel is applied onto a 3D-printed hollow microneedle structure. The microneedle array comprised of 12 hollow needles, with a hole diameter of 150 μm and a length of 1 mm, which are filled with the dual-network hydrogel. The hydrogel layer on the base had a thickness of approximately 300 μm. At Time=0 s, 50 μL of 0.1% methyl orange solution is dropped onto the tip of the microneedle patch, and photographs of the stained hydrogel are taken at Time=50 s and Time=100 s, indicating the diffusion efficiency of the double-network hydrogel as the diffusion layer. FIG. 10B shows the pH diffusion test in solution and in a hydrogel-filled microneedle. The pH values of different solutions with a pH range from 2-7 are measured using a pH meter. The purple dots represent the pH values measured directly in the solution, while the blue dots represent the pH values measured through microneedles filled with the double-network hydrogel, with a thickness of approximately 300 μm, after 200 s of stabilization following the addition of the solution with other pH levels, indicating the diffusion efficiency of the double-network hydrogel within the microneedle.

FIG. 11A shows the Bode plots (left) and the Warburg resistance-angular frequency plot (right) in hydrogels, respectively. FIG. 11B shows the Bode plots (left) and the Warburg resistance-angular frequency plot (right) in water, respectively. Substituting the value into the above formula, the diffusion coefficient of glucose in hydrogel and water can be estimated as:

$${D}_{hydrogel} = \frac{{8.314}^{2} \times {298.15}^{2}}{2 \times {0.00785}^{2} \times {1}^{4} \times {96485}^{4} \times {0.001}^{2} \times {12127}^{2}} = 3.9 \times {10}^{- 12} cm \cdot {s}^{- 1}$$

$${D}_{water} = \frac{{8.314}^{2} \times {298.15}^{2}}{2 \times {0.00785}^{2} \times {1}^{4} \times {96485}^{4} \times {0.001}^{2} \times {9376}^{2}} = 6.54 \times {10}^{- 12} cm \cdot {s}^{- 1}$$

Calculations show that glucose molecules have similar diffusion efficiencies in water and in the double-network hydrogel.

FIG. 12 shows the scanning electron microscope image of the IPN hydrogel filler layer. The IPN hydrogel layer functions as a filler within hollow microneedles, designed to establish a glucose diffusion pathway for an ISF-to-OECT glucose sensing system. The scanning electron microscope image illustrates the porous nature of the IPN hydrogel following the freeze-drying desiccation process. These pores are integral in facilitating the diffusion of glucose molecules, a critical mechanism essential for the sensor's operation.

FIG. 13. Shows the in vitro experiment setup of glucose monitoring in artificial ISF. The device for testing the concentration change of artificial interstitial fluid in vitro includes: i) an artificial ISF reservoir containing glucose solution on which the whole set of OECT-CGM can be placed stably, ii) A programmable syringe pump to push glucose solutions of different concentrations into the reservoir at a constant speed. iii) an agarose-thin-film-based artificial skin. By setting the syringe pump at different advancing speeds and filling the syringe with glucose solution with a gradient concentration, this experimental setup simulates different changes in glucose concentration in ISF.

FIG. 14 shows the stability test of the OECT-based glucose sensor during the two-week monitoring period, depicting a long-term stability test (14 days) of an OECT glucose sensor. The graph illustrates the response of the glucose sensor, measured as channel current variants (Alds), to a constant concentration of glucose solution (6 mM) at different time points. The Alds values have been normalized for comparison. It is evident that the current gradually decreases over time, reaching approximately 90% of the initial value by day 14. This decline in current indicates a decrease in the sensor's response to the same glucose concentration over the testing period, suggesting a decrease in long-term stability. n=10. Data are means±SD.

FIG. 15 is a photograph showing the in vivo continuous glucose measurement using the OECT-CGM system. This image provides an overview of the in vivo testing setup employed to validate the performance of the OECT-CGM system in real-time glucose concentration monitoring. The rat's dorsal area is carefully prepared by shaving it to facilitate the precise insertion of microneedles into the skin and to ensure optimal adhesion of the IPN hydrogel for effective OECT-CGM application. The highlighted area within the red dotted box represents the OECT-CGM device. In this configuration, glucose within the rat's tissue fluid naturally diffuses passively toward the OECT glucose sensor via the hydrogel situated within the hollow microneedle. Subsequently, the sensor data is acquired by the miniaturized PERfECT readout system and wirelessly transmitted to a mobile phone using Bluetooth Low Energy (BLE) technology, where it is recorded and analyzed. For the purpose of rat blood glucose level validation, blood samples are periodically collected from the rats' tail veins at 15-minute intervals over a duration of 2 hours and tested using a glucometer.

### DETAILED DESCRIPTION

Diabetes, especially in the case of type 2 diabetes, can be effectively managed and its progression significantly slowed or contained in its early stages. Therefore, regular blood glucose monitoring is essential in aiding diabetic patients in mitigating the impacts of diabetes to their health.

Traditional blood glucose monitoring is through invasive blood extraction techniques through, for example, “finger-pricking”, which may be inconvenient and may inflict slight pain in patients. As such, glucose monitoring methods with minimal intrusiveness is an increasingly popular alternative.

With improving technology, now blood glucose level may be detected not needing blood samples, but interstitial fluids may already suffice. As such, microneedle patches are developed, which greatly reduces the intrusiveness of the monitoring procedure, while also enabling continuous glucose monitoring (CGM), thereby providing real-time data and trends for patients and healthcare providers to make timely adjustments according to glucose level fluctuations on a timely basis.

However, existing technologies in the field still struggle with accuracy and calibration issues with sensors designed for interstitial fluid glucose readings. As presence of other substances in the interstitial fluid may interfere with glucose measurements, sensors with greater accuracy and calibration capabilities are needed. The organic electrochemical transistor-based continuous glucose monitoring system of the present invention solves these issues by integrating current regeneration, sensitivity adjustment and self-calibration capabilities through adjustable gate voltage and transconductance of the transistor, thereby ensuring the accuracy and reliability of the glucose level readings.

In addition, skin conditions such as skin allergies, scars and peripheral edema, which may arise from some more intrusive detection techniques, may affect the accuracy of the readings. The hydrogel-based membrane and microneedle patch continuous glucose monitoring system of the present invention is therefore formulated to minimize skin irritations and intrusion, therefore preventing the occurrence of these medical conditions, thereby not only ensuring a good accuracy of the glucose level readings, but also reducing any discomfort of the patients.

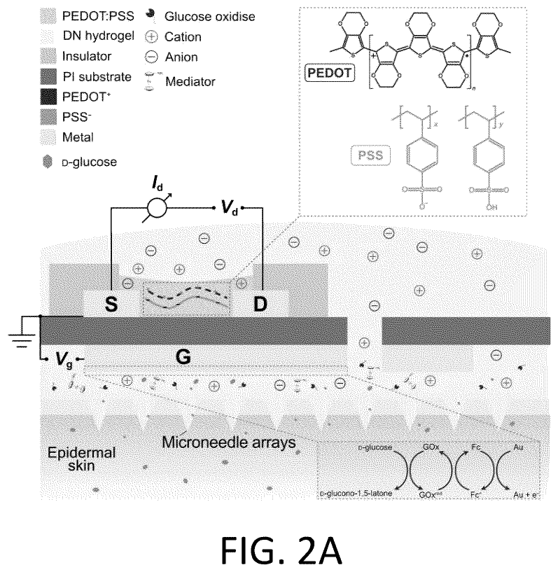

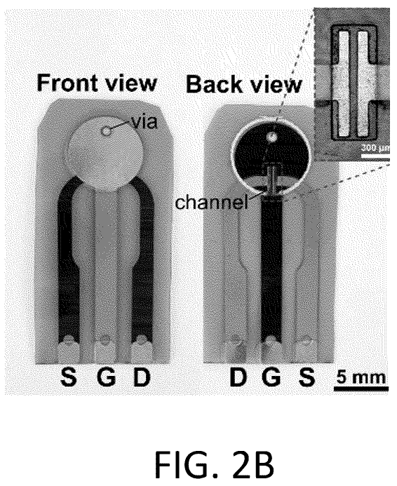

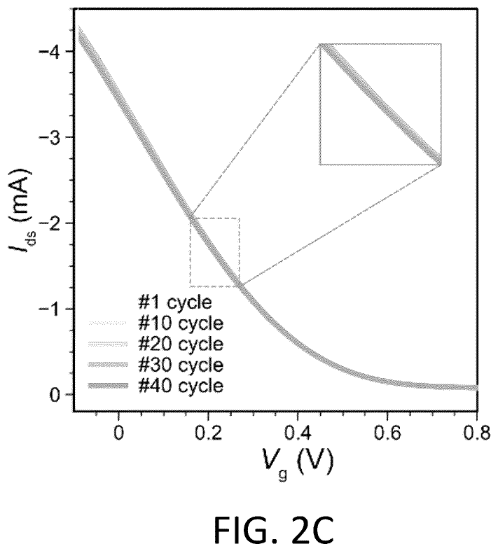

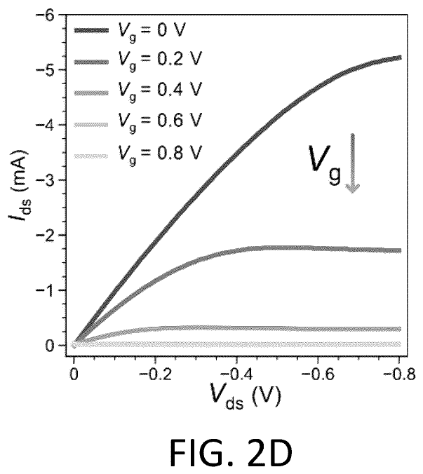

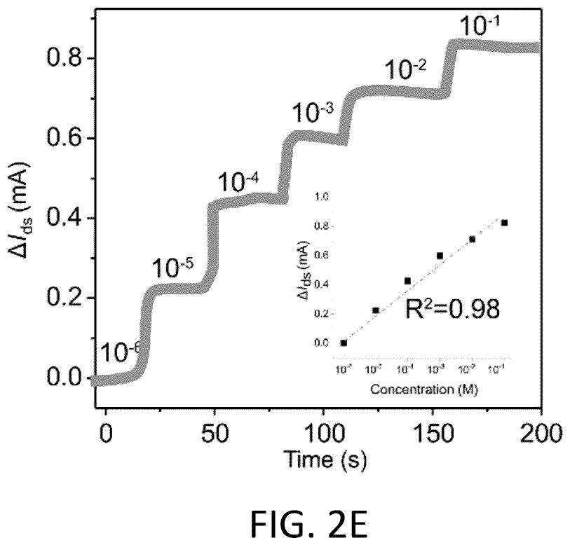

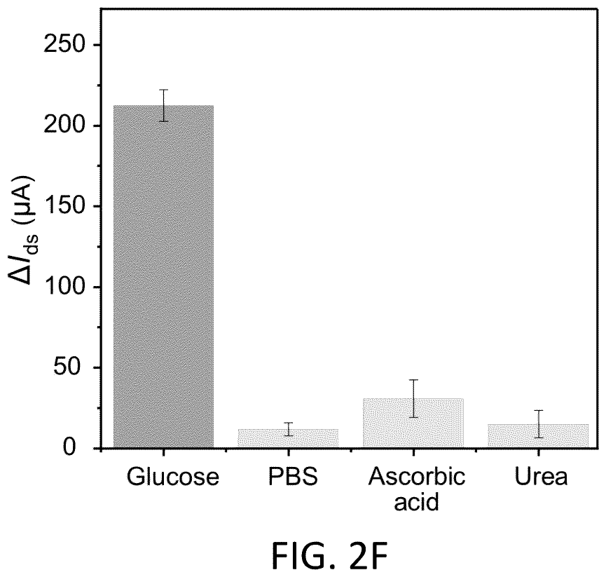

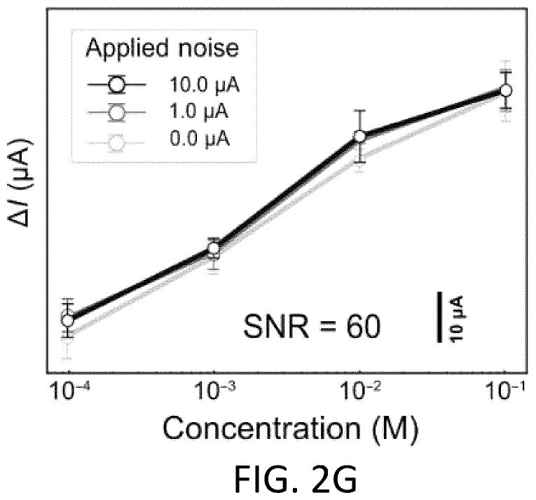

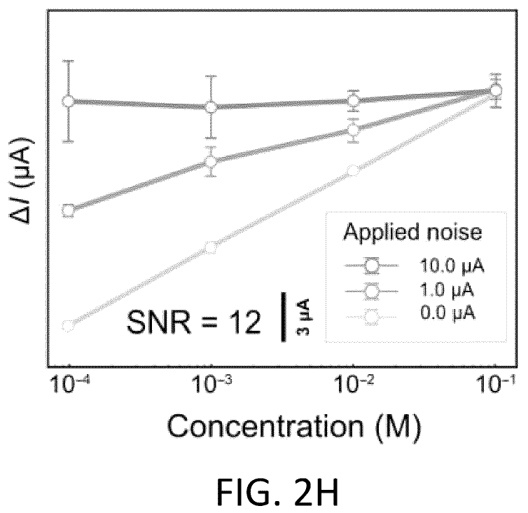

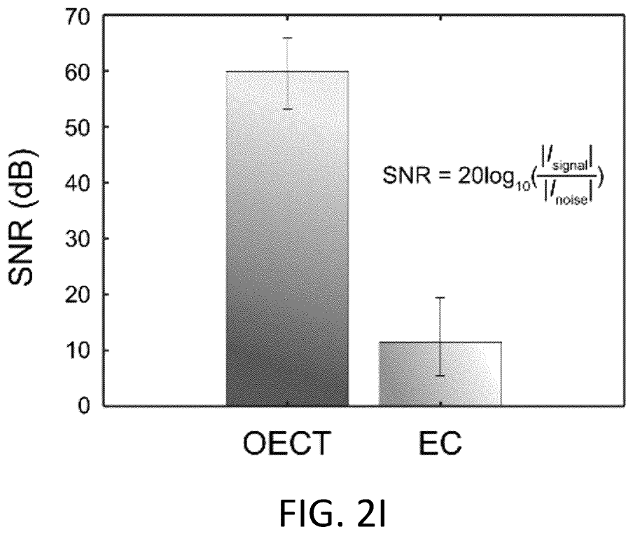

As illustrated in greater depths below, continuous glucose monitoring system of the present invention not only is able to minimize intrusion to the skin and thereby optimally mitigate discomfort to the patients, but also through integrating OECT into the system, the synergistic effects of the electrochemistry and transistor amplifiers also enhances the sensing quality and tunability of sensitivity of glucose level, while the signal-to-noise ratio is greatly improved to up to 60 dB as observed in FIG. 2I, a near 5-fold improvement to as compared to conventional electrochemical-based glucose sensors.

### EXAMPLES

### Example 1—Development and Proto-Typing of the OECT-CGM System

### 1.1 Design Principle of the OECT-CGM System

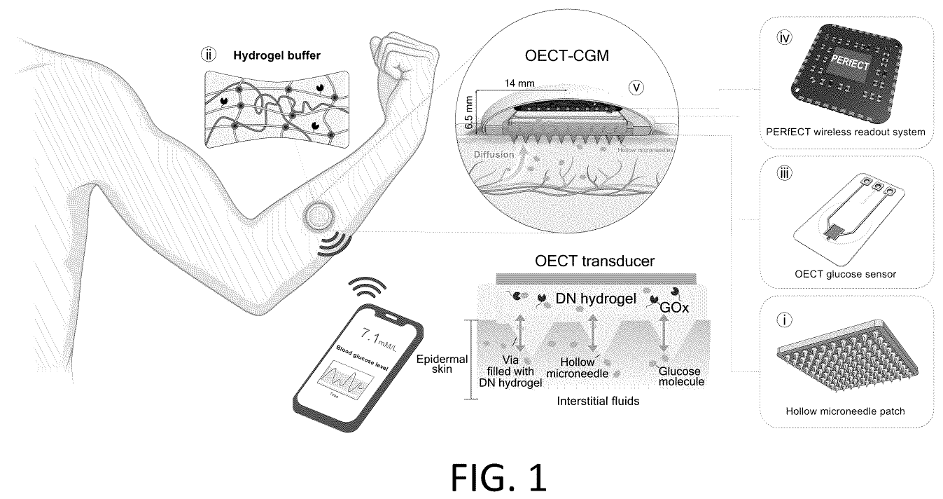

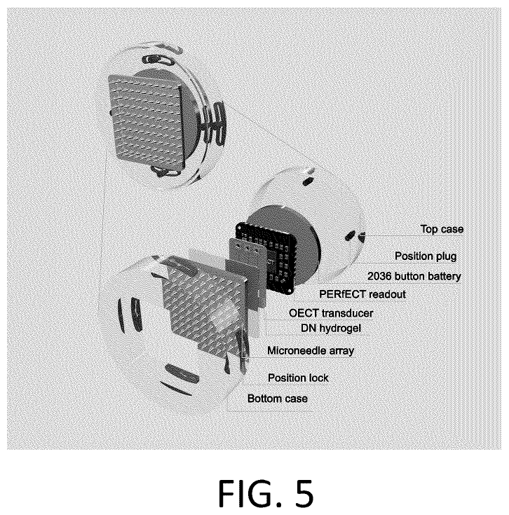

The coin-sized OECT-CGM system (FIGS. 1 and 5) is composed of five units: (i) a hollow microneedle patch, (ii) a tough and adhesive enzymatic hydrogel sensing membrane, (iii) an OECT-based glucose sensor, (iv) a miniaturized readout system, and (v) a 3D printed resin encapsulation case. The microneedle patch serves as a minimally-invasive channel that bridges the ISF and the OECT sensor. The stretchable and adhesive enzymatic hydrogel sensing membrane is sandwiched between the microneedle patch and the OECT glucose sensor to improve the interface adhesion and to buffer the motion artifacts. The glucose molecules in ISF passively diffuse to the surface of the OECT biosensor (through the microneedle and hydrogel) due to the concentration gradient. The adhesive, enzymatic hydrogel membrane is synthesized by constructing an interpenetrating double-network structure of polyacrylamide (PAAm) and Na⁺-Alginate. The synthesized hydrogel is further crosslinked with glucose oxidase (GOx) for glucose detection. The current of the OECT biosensor is recorded with a miniaturized and wireless readout system (FIG. 1) for data communication with mobile phones. A foldable flexible printed circuit board (fPCB) connector is implemented as the power line, facilitating an easy connection between the OECT sensor and the readout system. A stacking strategy is employed to reduce the physical dimensions (width and length) of the OECT-CGM system, thereby improving its wearability (FIG. 5).

### 1.2 Fabrication and Characterization of OECT Glucose Sensor

The OECT glucose sensor configuration is shown in FIGS. 2A and 2B. The OECT glucose sensor is fabricated on a flexible polyimide (PI) substrate (14 mm by 7 mm), featuring a round gate with a diameter of 3 mm, and a channel with a W/L of 1000 μm/100 μm.

The gate electrode is strategically situated on the backside of the PI substrate (FIG. 2B). This layout facilitates enzyme modification on the gate electrode and avoids undesired effects on the channel. The OECT showed typical transistor output curves working in depletion mode (FIG. 2C). The operational stability of the device is verified through cyclic transfer curves. A negligible shift is observed after the cyclic measurements (FIG. 2D), which underscores the sensor's commendable stability for biosensing.

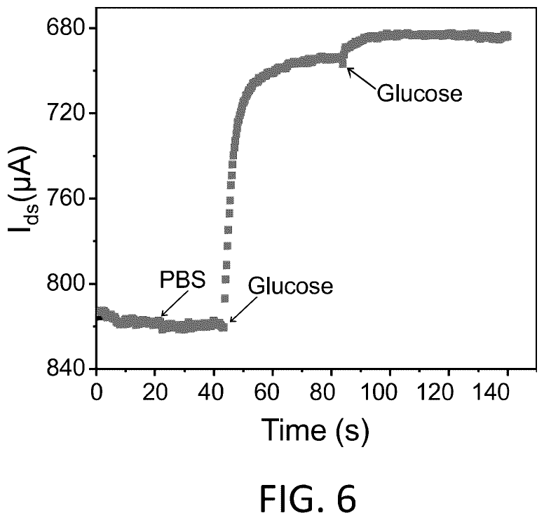

To make the OECT sensitive to glucose levels, GOx is mixed in a stretchable, interpenetrated double-network (IPN) hydrogel membrane. The sensing mechanism is illustrated in FIG. 2A, which includes the following processes: i) Glucose Oxidation: When glucose is presented, it is oxidized by the GOx embedded in the enzymatic hydrogel, producing gluconolactone and reducing GOx; ii) Enzyme Regeneration: The reduced form of GOx is oxidized by the mediator ferrocene and regenerated to the oxidized form. In turn, the ferrocene mediator becomes reduced; iii) Mediator Oxidation: The reduced form of the ferrocene mediator is then oxidized back to ferrocene at the gate electrode surface. The series of reactions mentioned above yields a faradic current (I_{g}), leading to a change in the effective gate voltage (V_{g}). This alteration, albeit small, is then amplified by the OECT, resulting in a substantial change in the drain-source current (I_{ds}) (FIG. 6). Such an on-site signal amplification process strengthens the tolerance to the line noise, thereby leading to a high signal-to-noise ratio (SNR).

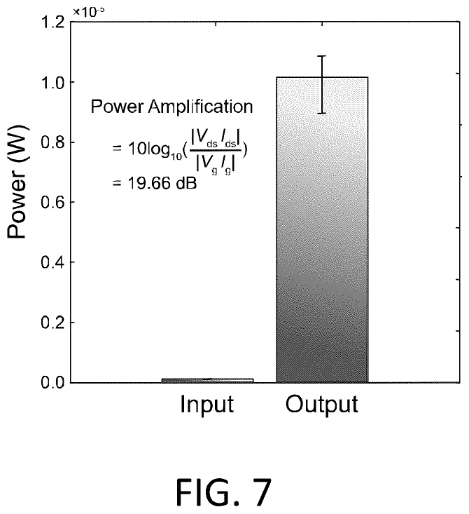

The fabricated OECT biosensor demonstrated a linear response to a wide range of glucose concentrations, ranging from 106 mM to 10⁻¹ mM (FIG. 2E). Furthermore, interference experiments revealed a negligible sensor response to common interfering substances, such as ascorbic acid and urea, attributable to the high selectivity of GOx and confirmed that those inherence substances have minor effect on the response (I_{ds}) of OECTs (FIG. 2F). The above results indicate the capability of the OECT glucose biosensor to be used in harsh environments for bodily liquid analysis. Compared to conventional electrochemical-based glucose sensors, OECT-based glucose sensors exhibit enhanced anti-noise capability due to the inherent high I_{ds} (FIGS. 2G and 2H). The high I_{ds} of the OECT sensor is derived from the integration and amplification of the gate signal (I_{g}), making it highly practical in situations where signal transmission is susceptible to environmental noise (FIG. 7). FIG. 2I provides a comparative analysis of the SNR between a traditional electrochemical-based glucose sensor and an OECT-based glucose sensor, confirming the superior value of the later one.

### 1.3 Tunable Sensitivity, Current Regeneration and Self-Calibration Ability of OECT Glucose Sensor

Tunable sensitivity, current regeneration and self-calibration ability have been long pursued by current electrochemical glucose biosensors for improved diabetes management, though their simultaneous realization remains elusive. Firstly, tunable sensitivity allows for focused monitoring of patient-specific glucose ranges (between 1 mM to 20 mM) for precise diabetes management and personalized medication. Secondly, the current regeneration ability is necessary to prolong the sensor's lifetime by mitigating signal degradation over time. Thirdly, self-calibration capability is a critical function to simplify the user experience by removing the need for manual calibration prior to each use. Despite significant advances in the field, the concurrent achievement of these functions within a single biosensor system is yet to be accomplished.

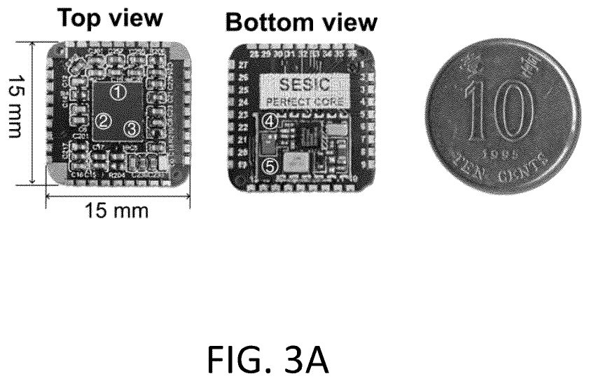

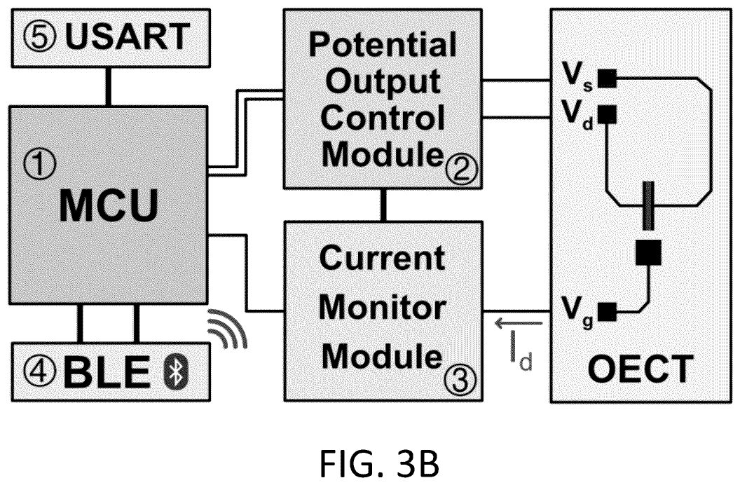

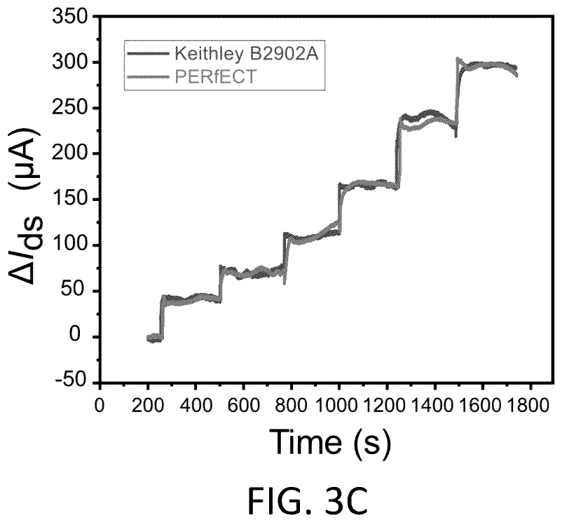

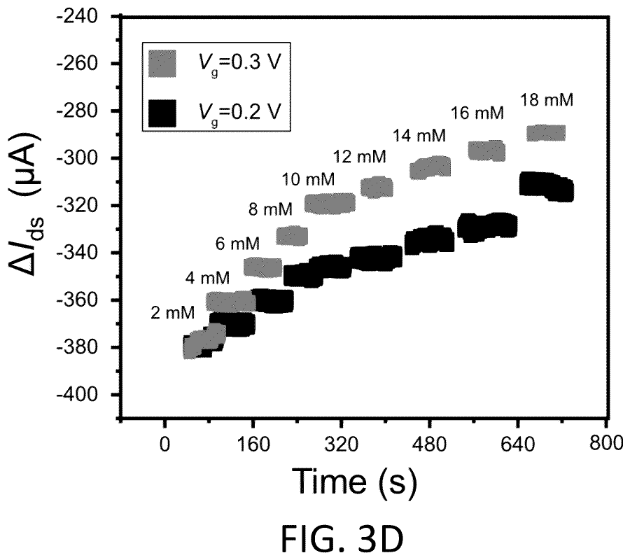

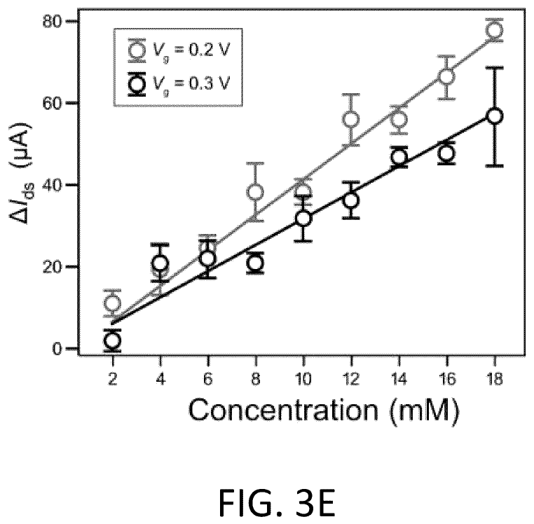

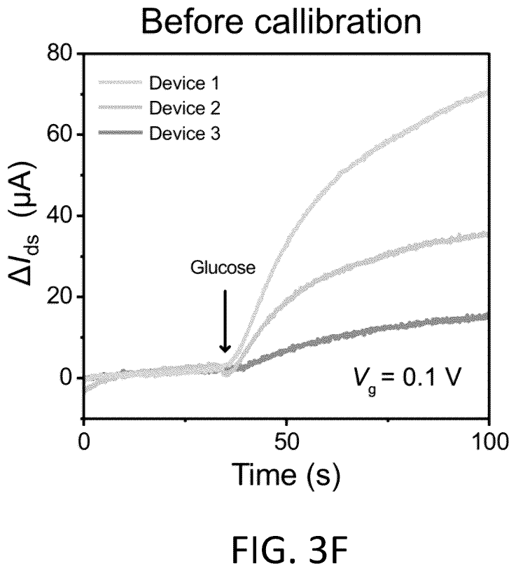

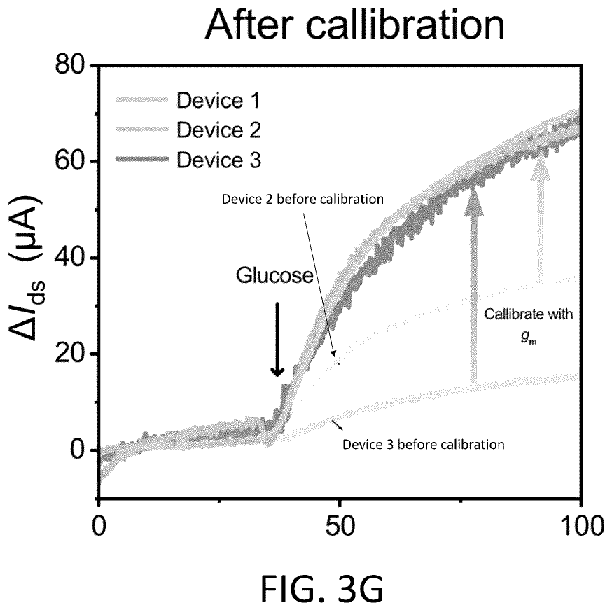

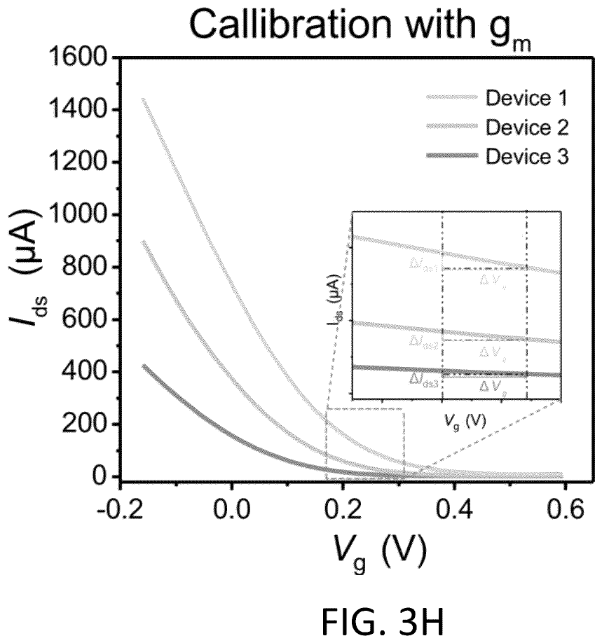

The OECT glucose biosensor allows concurrent achievement of those new features in one device. To demonstrate those functions, the sensor is measured with a coin-sized, precise PERFECT readout system (dimension of 1.5 cm×1.5 cm×0.2 cm, weight of 0.4 grams) so that the module is integrable with smart wearables (FIGS. 3A and 3B). The logic diagram of the PERFECT system is shown in FIG. 3B. The potential control module takes commands from the microcontroller unit (MCU) to control the V_{g} and V_{ds} with steps down to 2 mV for precise characterization of the dynamic behaviors of the OECT sensor. The current monitor module can read the I_{ds} with the detection limit down to 1 nA, endowing PERFECT with benchmarkable resolution in both applying V_{g} and V_{ds} and reading las when compared with the lab-used source measure unit (SMU) (FIG. 3C).

The tunable sensitivity is achieved by changing the gate voltage of the OECTs. As shown in FIGS. 3D and E, alternating the V_{ds} from 0.3 V to 0.2 V improved a linear sensitivity from 0.0035 μA·mM⁻¹ to 0.0042 μA·mM⁻¹. This feature is achievable because the sensitivity (transconductance) of three-terminal OECTs has a linear relationship with V_{g}, which is governed by the following equation (1):

$${g}_{m} = \frac{wd}{l} μ C * ( {V}_{th} - {V}_{g} ) \qquad ( 1 )$$

where gₘ is the transconductance, w is the channel width, d is the channel thickness, 1 is the channel length, μ is the charge carrier mobility, C* is the volumetric capacitance, Vₜₕ is the threshold voltage, and V_{g} is the gate voltage. OECTs owns the highest μC* among biotransistors, therefore permitting the highest sensitivity and SNR, derived from the power amplification ability of OECT as a biotransistor (FIG. 7).

Current regeneration is achieved by decreasing the V_{g} of OECTs which, in turn, increases the I_{ds} because the adopted PEDOT:PSS OECTs is a p-type transistor working in depletion mode. However, as discussed above, changing the V_{g} will also affect the sensitivity. Therefore, self-calibration ability of the system is needed. This function is achieved by normalizing the I_{ds} (FIG. 3F-H) with the transconductance values. As shown in FIG. 3G, identical current responses to glucose levels are obtained after calibration (as ΔI_{ds}=Δgₘ·ΔV) to compensate for device-to-device variations. The current regeneration and self-calibration ability can avoid current loss over increased implantation time, which holds the potential to improve the anti-noise ability and long-term stability during practical use.

### 1.4 Prototyping of the OECT-CGM System

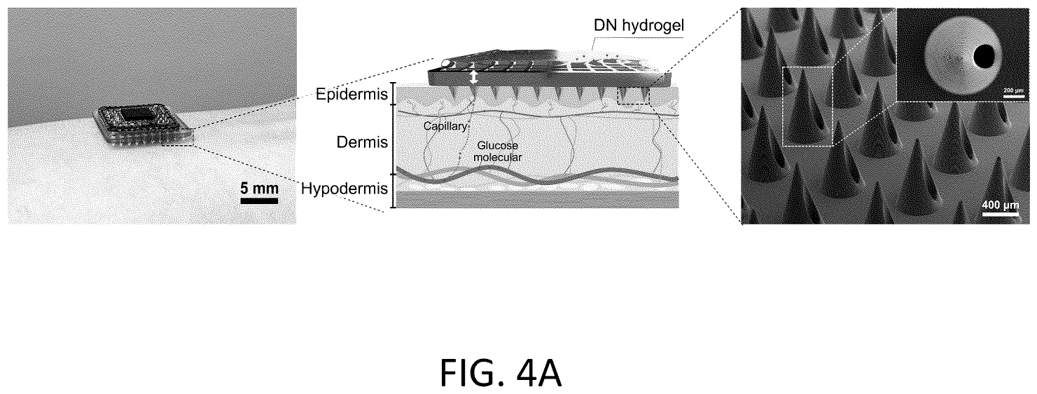

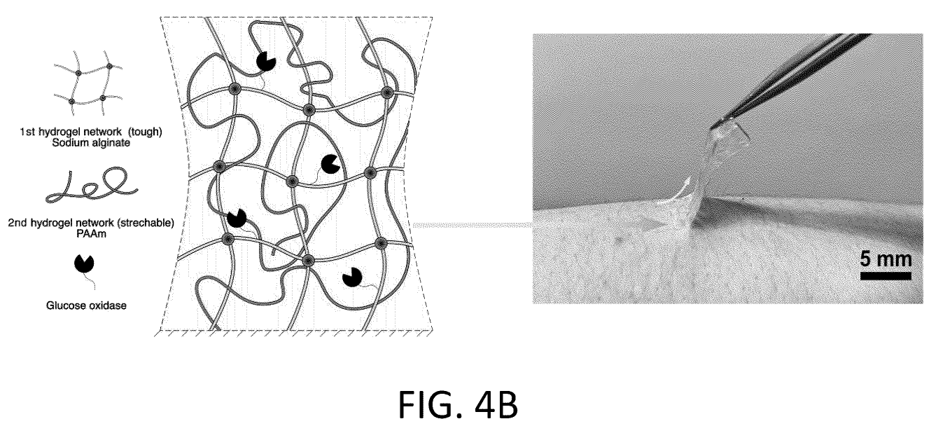

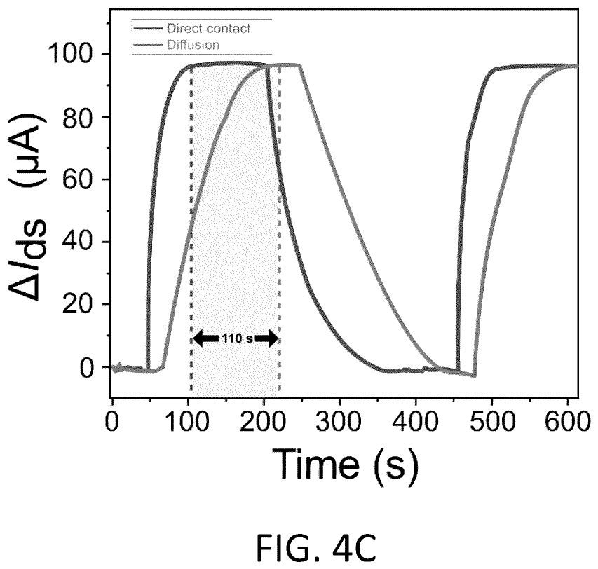

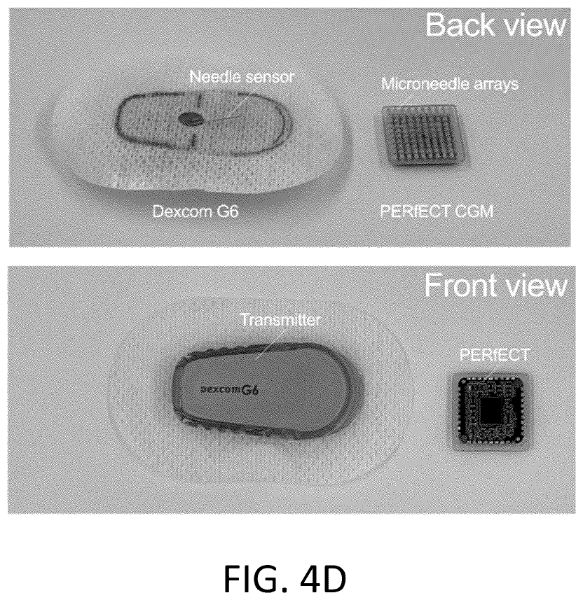

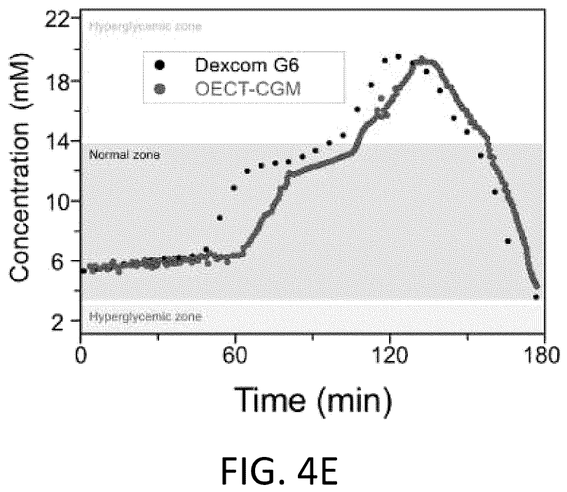

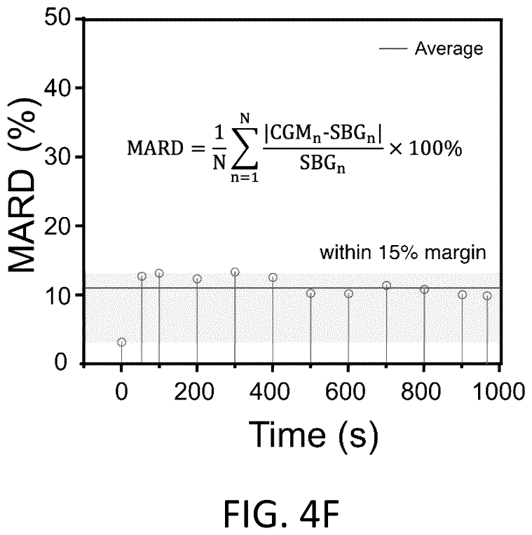

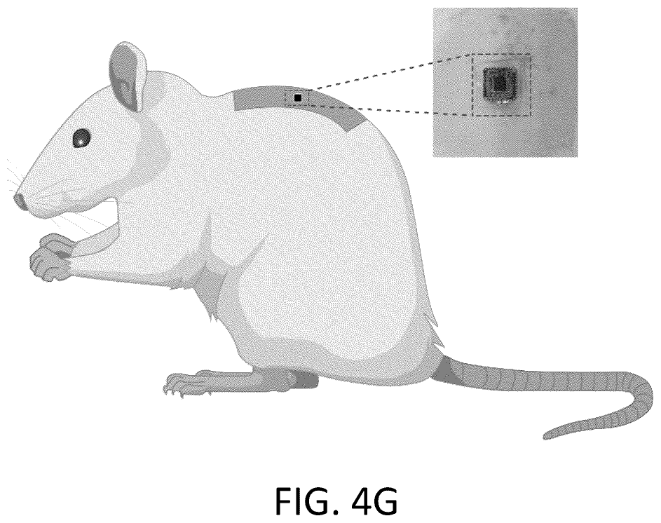

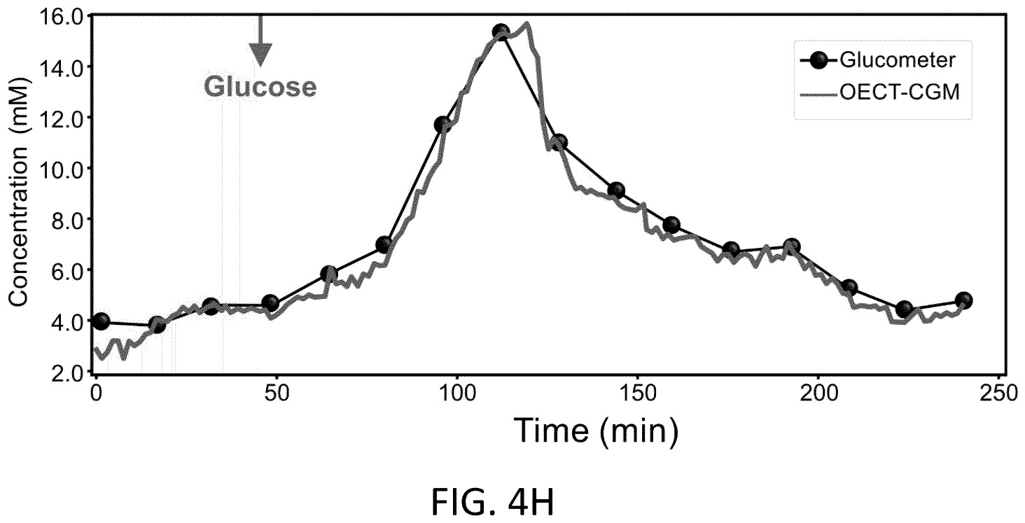

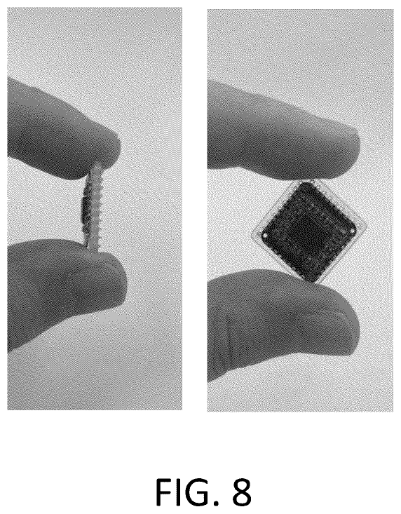

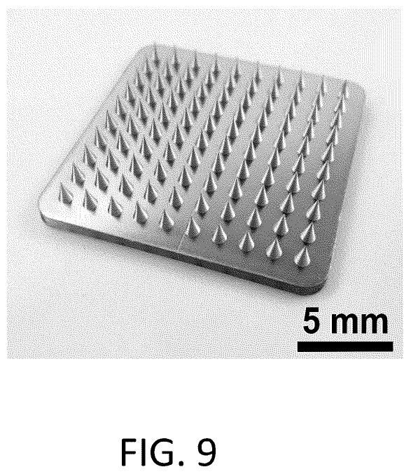

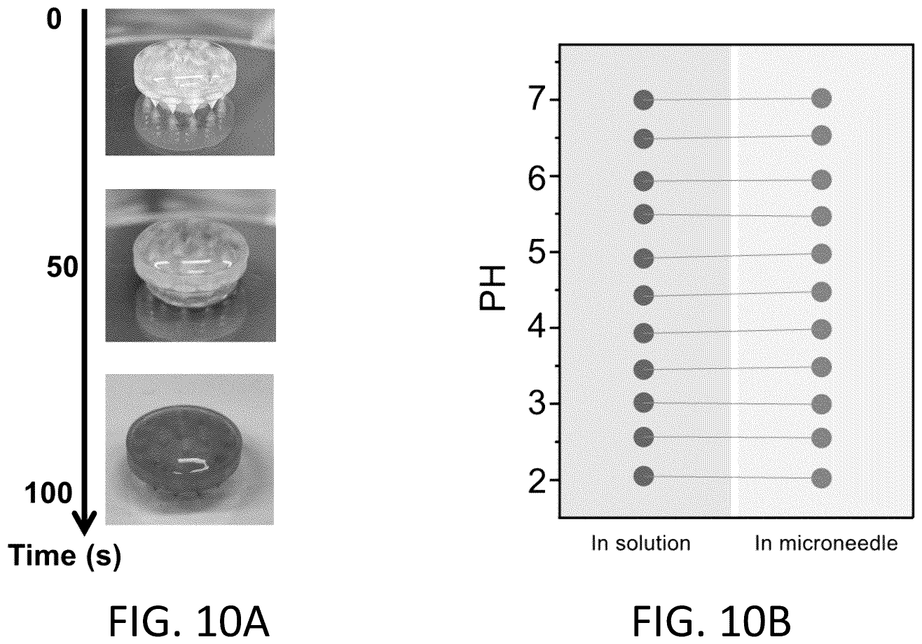

Microneedle for minimally-invasive ISF sampling: One of the main obstacles against the wide adoption of the current CGM system is the pain associated with the implantation process. Recent works have proved that decreasing the needle length of microneedles can help reduce pain in human subjects. Microneedles, and specifically microneedle arrays, provide the ability to sample biological fluid with less pain provided that the needles do not penetrate the skin to the depth of the dermis where capillaries and most nerve endings reside. By not penetrating to this depth, the biological fluid sampled is not blood, but ISF residing in the viable epidermis (FIG. 4A). For the OECT-CGM system, the microneedle length is set to 1.0 mm (FIG. 4A). For better penetration of the skin, a sharpened tapered shape of microneedle in 10×10 arrays is employed, with the taper angle of 15 degrees. The efficiency of this structure in penetrating the skin has been previously reported. The size of the microneedle array is designed as 10 mm×10 mm×2.2 mm to be compatible with the CGM readout system (FIG. 8). Furthermore, the surface properties of the resin-based 3D printed microneedle array can be modified by metallizing the needle tips (FIG. 9).

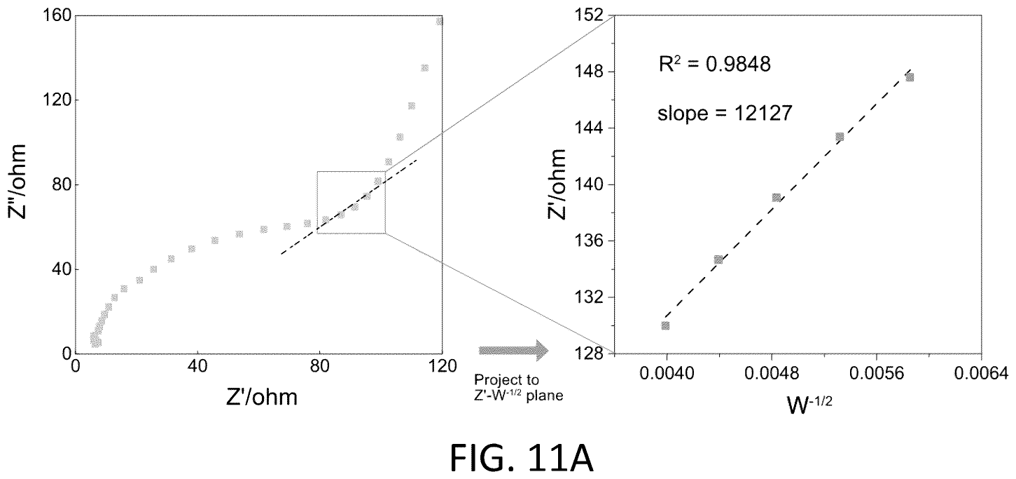

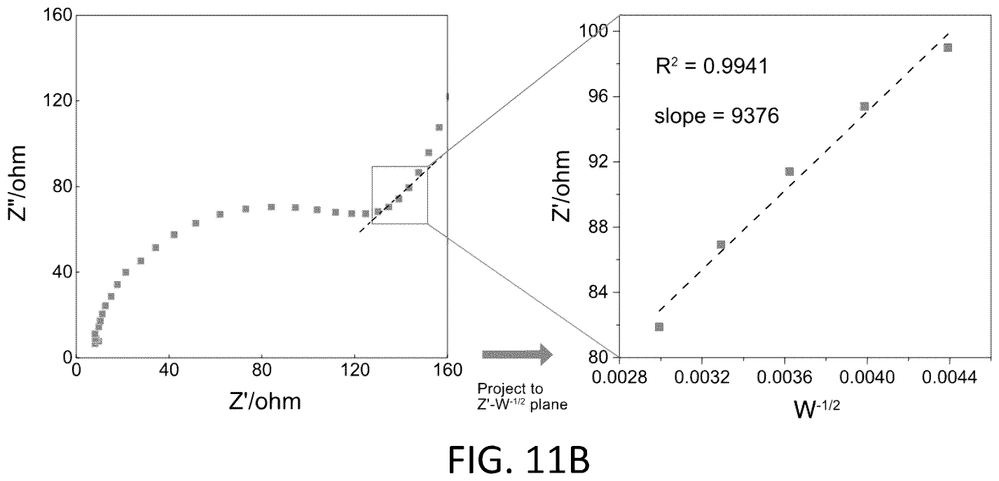

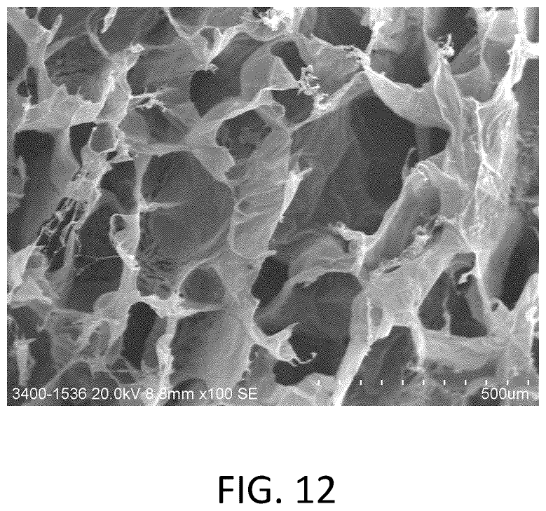

Hydrogel filler to promote glucose diffusion from ISF to OECT sensor: glucose molecules in the ISF can diffuse to the sensor passively via the hollow microneedle because of the concentration gradient. Because the glucose will keep being consumed on the gate electrode of the OECT glucose sensor, there is a consistent concentration gradient between the IFS and the sensor in the vertical direction, driving the diffusion of the glucose through the microneedle holes to the electrode (FIG. 4A). However, clogging frequently happened because of the wound healing at the tips of the microneedles. A hydrogel is thus selected and used as a filler in the hollow microneedles to prevent wound healing, while maintaining efficient diffusion of target molecules to the sensor (FIGS. 11 and 12). To evaluate the diffusion efficiency of the hydrogel, the glucose diffusion coefficient is measured based on the mass-controlled Warburg element through EIS analysis (FIGS. 11 and Para. [0042] below). The Warburg impedance depends on several factors, including the concentration of the active material, the diffusion coefficients, and the number of mobile electrons. Warburg impedance, also known as the Warburg coefficient (σ) can be expressed as follow, engendering the diffusion coefficient term:

$$σ = \frac{RT}{{F}^{2} A \sqrt{2}} ( \frac{1}{{D}_{O}^{1 / 2} {C}_{O}^{*}} + \frac{1}{{D}_{R}^{1 / 2} {C}_{R}^{*}} ) \qquad ( 2 )$$

where R is the gas constant (8.314 J·mol⁻¹·K⁻¹), T is the temperature (in Kelvin), F is the Faraday's constant (96485 C·mol⁻¹), A is the electrode surface area (cm²), C_{O} is the concentration of the oxidized species (in mol), C_{R} is the concentration of the reduced species (mol), D_{O} is the diffusion coefficient of the oxidized species (cm·s⁻¹), and D_{R} is the diffusion coefficient of the oxidized species (cm·s⁻¹).

To evaluate the diffusion efficiency of the proposed double-network hydrogel as a passive diffusion layer, electrochemical impedance spectroscopy (EIS) is deployed to measure the impedance of the proposed system as a function of frequency. The glucose diffusion coefficient from the hydrogel layer to near the electrode can be estimated using the Warburg impedance, a characteristic feature of EIS spectra at low frequencies. The diffusion of glucose molecules near the electrode can be described as Fick's second law of diffusion:

$$\frac{\partial C}{\partial t} = D \frac{\partial {C}^{2}}{\partial {x}^{2}}$$

where C is glucose concentration, t is the time, x is the distance, and D is the diffusion coefficient. To estimate D, the Warburg impedance needs to be observed in EIS. Warburg impedance is usually observed in the low-frequency range. The relationship between the diffusion coefficient of glucose and the slope of the Warburg impedance line can be expressed as:

$$D = \frac{{R}^{2} {T}^{2}}{2 {A}^{2} {n}^{4} {F}^{4} {C}^{2} {σ}^{2}}$$

where D is the diffusion coefficient, R is the ideal gas constant, T is the absolute temperature, A is the area of the electrode, n is the number of electrons transferred per mole participating in the electrode reaction, F is Faraday's constant, C is the concentration of the glucose concentration, σ is the Warburg coefficient, which is the slope of Warburg resistance line in the

$${Z}^{′} - {ω}^{- \frac{1}{2}}$$

plane.

By controlling the compositions and thickness of the hydrogel membrane, a glucose coefficient (3.90×10⁻¹² cm·s⁻¹) close to that of water 6.54×10⁻¹² cm·s⁻¹ is obtained, thanks to the high porosity and hydrophilicity of the hydrogel. Despite a similar sensitivity obtained using a hydrogel filler, a response delay of 120 seconds is also noted compared to the reference sensor that is in direct contact with the ISF. This delay is observed regardless of glucose concentrations (1 mM to 10 mM), which is in line with the microneedle-based sensors and attributable to the glucose diffusion process from the ISF to the OECT sensor (FIG. 4F).

Tough, sticky hydrogel to improve adhesion between skin and the OECT-CGM: robust adhesion between the sensor and skin is a crucial factor to improve the durability and wearability of CGMs. Toward this goal, the introduced hydrogel is further engineered to be tough and adhesive by hybridizing the IPN-hydrogel with a semipermeable bioadhesive elastomer. The IPN-hydrogel consists of a tough sodium alginate first network and a soft PAAm second network, functionalized with a mixture of GOx (FIG. 4B). The tough and adhesive nature of the IPN-hydrogel-elastomer hybrid ensures that the system remains in place on the skin even under strain and twist conditions. Additionally, mixing the GOx in the IPN-hydrogel provides a facile way for glucose detection without complicated enzymatic modification procedures on the electrodes. The IPN-hydrogel-based sampling and sensing system is thus expected to serve as a promising solution to improve the reliability and wearability of wearable CGM systems.

To validate the efficacy of the fully integrated OECT-CGM system, it is compared with a reference CGM system (Dexcom G6). The reference device consists of 4 parts, the needle-based sensor with a length of 2 cm, the adhesive patch, the supporting frame, and a wireless data transmitter. The OECT-CGM system proposed here consists of five parts: A microneedle array with a needle length of 1.0 mm, an IPN-hydrogel-elastomer hybrid for glucose detection and adhesion, an OECT transducer, PERFECT readout system and an fPCB-based connecter with magnetic aligner and metal contacts to case the integration with a smartwatch.

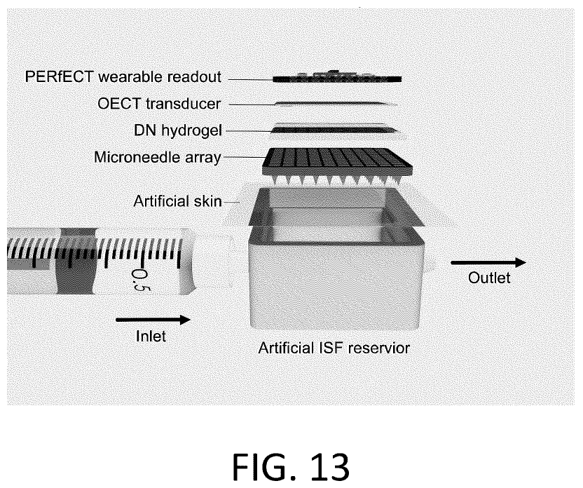

An in vitro experiment is conducted to benchmark the results of OECT-CGM and the reference device (FIG. 13). An artificial skin made of elastomer thin films is used to validate the microneedle penetration effect. During the experiments, the glucose levels are dynamically controlled in the chamber with a pump and monitored the values with both the OECT-CGM system and the reference device. During the 180-minute test, the concentration is cyclically changed from clinically relevant values (3 mM to 20 mM). The OECT-CGM system shows comparable results to the reference device (FIG. 4H), but with a smaller size (1.5 cm×1.5 cm) and weight (FIG. 4D). A response delay of ˜8 min is observed in the OECT-CGM system before reaching the equilibrium state, attributable to the diffusion delay of glucose molecules in the IPN-hydrogels. The OECT-CGM exhibits an acceptable mean absolute relative difference (MARD) value of 15%, demonstrating its potential for future practical use (FIG. 4F).

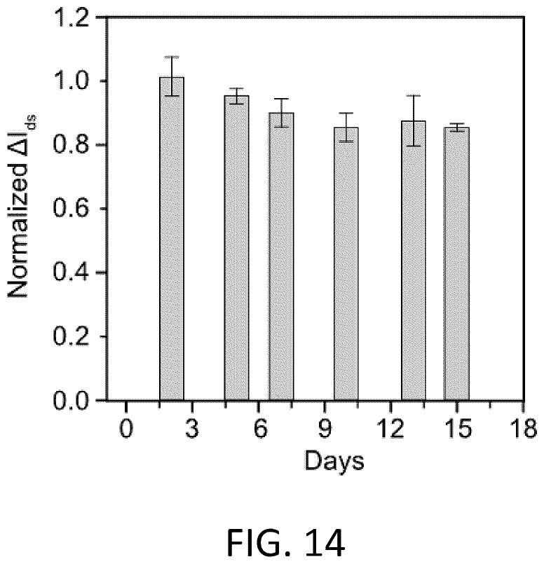

The current regeneration ability of the OECT-CGM system is further validated. As shown in FIG. 14, the current variation of the device gradually decreased from 355 μA to 322 μA after two weeks of continuous monitoring. By decreasing the gate voltage from 0.3 V to 0.2 V, the current regenerated from 322 μA to 345 μA under 6 mM glucose concentration. Besides, after a self-calibration process with the in-situ measured transconductance values, identical sensitivity is re-obtained but with a higher current which is immune to ambient noise.

### 1.5 In Vivo Continuous Glucose Measurement Using the OECT-CGM System

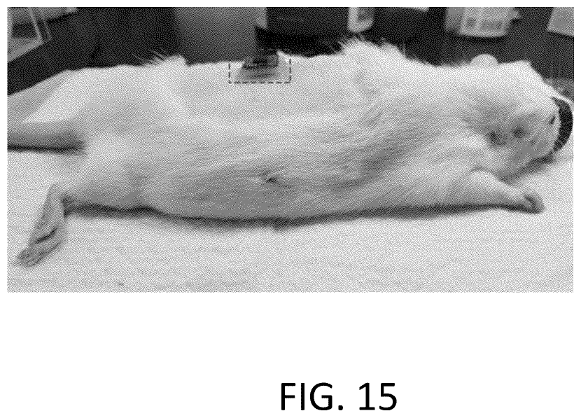

In order to validate the continuous glucose monitoring (CGM) capabilities of the proposed OECT-CGM, an in vivo experiment is conducted involving rats, as depicted in FIG. 4G. To facilitate effective monitoring, the dorsal region is selected as the application site, as it allowed for the appropriate insertion of microneedles into the dorsal area of the rats, ensuring the stable operation of the OECT-CGM within this region, as illustrated in FIG. 15. To induce hyperglycemic conditions in the rat model, glucose solutions are administered to manipulate the blood glucose levels of the rats. Once the blood glucose levels of the rats reached their peak, the animals relied on their own natural glucose regulation mechanisms to lower their blood glucose levels back to normal conditions. The blood glucose levels in the rats peaked at approximately 15.4 mM/L approximately one hour after the administration of the glucose solution.

The OECT-CGM continuously measured the glucose levels at intervals of 90 seconds. Furthermore, at 15-minute intervals, blood samples are collected from the rats' tail veins, and commercial blood glucose meters are utilized to measure their blood glucose levels for comparison. This data collection process continued for a duration of two hours. The results indicate a strong correlation between the OECT-CGM glucose sensor and the commercial glucometer. Notably, the peak values of these two measurements occurred in a relatively synchronous fashion (t=110 min). This observation highlights the feasibility and efficacy of the present OECT-CGM in vivo applications. It is worth mentioning that a slight delay of approximately 15 minutes is observed, which can be attributed to the diffusion of glucose from the bloodstream into the tissue fluid, followed by its subsequent diffusion from the tissue fluid through the hydrogel layer to reach the sensor, as elucidated in FIG. 4H.

### Example 2—Discussion

In conclusion, the OECT-CGM system describe in this work represents a noteworthy step forward in the field of wearable CGM. By integrating OECTs as glucose biosensors and microneedle arrays as minimally invasive sampling techniques, the system offers three key features that show promise for improving glucose monitoring. Firstly, it provides an adjustable linear region, offering enhanced sensitivity and customizable measurements. This feature holds the potential for accurate monitoring in real scenarios. Secondly, the OECT-CGM system demonstrates current regeneration ability, achieved by adjusting the gate voltage. This capability allows the device to recover its performance by releasing trapped ions or molecules, addressing concerns related to performance degradation over time and in challenging environments. Lastly, the system incorporates self-calibration ability, enabling accurate glucose measurements without the need for external calibration.

Furthermore, the OECT-CGM system exhibits stability through various mechanisms. The current regeneration ability, combined with the stability of the GOx mixed in the tough hydrogel, as well as acting as a mechanically adhesive buffer layer, contributes to the overall system's stability. Additionally, the microneedle arrays act as a bridge between the interior environment and exteriorly deployed biosensor, minimizing foreign body reactions and potential biofouling commonly encountered in CGM.

### Example 3—Materials and Methods

### 3.1 Materials

PEDOT:PSS aqueous suspension (Clevios PH1000) is purchased from Heraeus Electronic Material (USA). Glycerol, dodecylbenzene sulfonic acid (DBSA), sodium chloride, (3-glycidyloxypropyl) trimethoxysilane (GOPS), acrylamide (AAm), calcium chloride (CaCl₂)) and 3-(trimethoxysilyl) propyl methacrylate (TMSPMA) are purchased from the Sigma-Aldrich (USA). Hydroxy-4′-(2-hydroxyethoxy)-2-methylpropiophenone (Irgacure 2959), glutaraldehyde, ferrocene and GOx and chitosan are provided by Aladdin Co. (Shanghai, China). The PI thin film is obtained from the DuPont Co. (U.S.A.). The biocompatible adhesive polyurethane is provided by 3M (U.S.A.). Unless otherwise specified, the chemicals in the current work are used without further purification.

### 3.2 Animals

The animal work is approved by the Animal Ethics Committee of Guangzhou Medical University and conducted in accordance with relevant guidelines. Female SD rats (300 to 350 g) are purchased from the Guangdong Medical Laboratory Animal Center and acclimatized in an approved animal facility.

### 3.3 Microneedle Fabrication

To fabricate the hollow microneedle patch, a high-resolution 3D printer (S240 from Boston Micro Fabrication) is used. This printer utilizes ultra-precision stercolithography (uPSL) technology, which enables printing with a precision of up to 10 μm in both the lateral and vertical directions. The microneedle structures are designed using computer-aided design (CAD) software and then printed using a photopolymer resin. The printed structures are then post-processed by curing them in a UV chamber and rinsing them with isopropyl alcohol to remove any uncured resin. The microneedle patch is tested for mechanical strength and skin penetration ability using an in vitro skin model.

### 3.4 OECT Sensor Fabrication

To fabricate the OECT on a flexible PI substrate, the process started with constructing the source/drain/gate electrodes by depositing a layer of gold thin film on above, the pattern of which is defined by a metal shadow mask. Then the active channel layer, PEDOT:PSS, is deposited between the source and drain electrodes by using inkjet printing with a minimal droplet size of 1 pL. The printable PEDOT:PSS ink is prepared by firstly stirred for 3 minutes and then mixed with GOPS (1w/w. %), glycerol (5 v/v. %) and DBSA (0.1 v/v. %) with a Vortex (MX-S). The addition of glycerol is to increase the film conductivity. DBSA is added to facilitate the wetting property of films on substrates. Before printing, the mixed suspension is filtered with a polytetrafluoroethylene (PTFE) membrane (aperture size of 0.45 μm) to remove aggregates to avoid clogging in the nozzle. Next, the insulation layer of UV-cured resin is also deposited using inkjet printing system (SWA3060, Yiwu Yangtian Electronic Techonlogy Co., Ltd, Yiwu, China) and cured in situ using the UV lamp (wave length: 345 nm) attached to the printer. To complete the OECT structure, a tough IPN hydrogel composed of PAAm/sodium alginate double-network is prepared to bridge the gate electrode and the channel.

To synthesize the tough IPN hydrogel, 2% (w/w) sodium alginate, 13% (w/w) AAm, 0.015% (w/w) MBAA and 0.24% (w/w) Irgacure 2959 are dissolved in deionized water. The mixture is then centrifuged at 9000 rpm to remove air bubbles. The formation of the PAAm network can be formed by curing the solution in ultraviolet light (UV) chamber (364 nm, 10 W power) for 60 min. The second network is formed by crosslinking the sodium alginate via immersing the previous gel in 1M CaCl₂) solution for 24 hours to reach an equilibrium state.

To endow the system with the specificity for glucose sensing, the enzymatic hydrogel is prepared by embedding the GOx and ferrocene within the tough IPN hydrogel, in which the GOx can selectively react with the glucose, ferrocene act as the redox mediator. To do this, the IPN hydrogel is firstly freeze dried for 48 hours to obtain the double-network scaffold. After swelling the scaffold in an aqueous solution containing GOx (500 units/mL), ferrocene (10 mM), and glutaraldehyde (0.3% (w/w)) overnight, the enzymatic tough hydrogel can be obtained.

To make the IPN hydrogel adhesive, the hydrogel is hybridized with a bio-adhesive elastomer thin film (provided from 3M). The elastomer thin film is perforated with a hole punch (200 μm in diameter) to facilitate the exchange of ISF among body, hydrogel and OECT sensor. The adhesive elastomer is firstly treated with TMSPMA solution (100 ml deionized water, 10 μL of acetic acid with pH 3.5 and 2 wt. % of TMSPMA), then washed with ethanol and dried. The gel precursor is carefully dropped on the TMSPMA grafted elastomer to avoid bubbles and cure under UV irradiation for 60 mins. The IPN hydrogel formation follows the same procedure as described above. The grafted TMSPMA tends to interconnect the IPN hydrogel and elastomer to form the hybrid that can firmly bond to the human skin.

### 3.6 Miniaturized Readout System Fabrication and Evaluations

To shrink down the size of the readout system, the recently developed miniaturized OECT readout system PERFECT is modified to meet the specific requires for CGM. The PERFECT system is customized with firmware and designed to include a customized MCU, Bluetooth Low Energy (BLE) unit, and other necessary components, all soldered onto a printed circuit board (PCB) using standard fabrication processes. The readout system is then connected to the OECT glucose sensor using an fPCB connector. The typical power consumption of the PERFECT system when sampling at a rate of 1 sample per second (SaPS) is measured to be 1 mW using Keithley B2902B. The performance of the modified PERFECT system is evaluated by conducting in vitro experiments using simulated glucose solutions, and the accuracy and precision of the system are analyzed using statistical methods.

### 3.7 Integration Strategy

To achieve the integration of the various components, a meticulous approach is taken. The microneedle patch, produced through high-resolution 3D printing, is seamlessly linked to the OECT sensor. This is accomplished by utilizing an fPCB connector, which ensured reliable electrical connectivity between the sensor and the miniaturized readout system. The readout system itself underwent customization, incorporating an MCU, a BLE unit, and other crucial components. This tailored design facilitated a smooth and optimized interface with the OECT sensor. By employing efficient connection techniques and carefully assembling these components, a compact and fully integrated CGM system is successfully realized, enabling continuous glucose monitoring with utmost user-friendliness and operational efficiency.

### 3.8 Anti-Interference Tests

In the pursuit of rigorous assessment of the selectivity of the OECT-based glucose sensor, an integral component of the compact OECT-CGM system, a comprehensive anti-interference test is executed. This evaluation aimed to determine the sensor's ability to discern and accurately measure glucose levels amidst the presence of various interfering substances that could conceivably coexist in the human body.

To simulate realistic conditions and encompass a broad spectrum of potential interfering agents, four representative substances are selected, namely uric acid, ascorbic acid, acetaminophen, and lactic acid. These substances are introduced into the test container at a concentration of 1M, emulating concentrations relevant to in vivo scenarios.

To ensure precision and control throughout the experimentation process, a sophisticated programmable injection pump (XMSP-2CI, Ximai Nanotech, China) is employed. This enabled us to modulate the concentration of the interference substances within the test container systematically, achieved by a step motor that controls the movement of the syringe down to 10 μm. By incorporating such precision in this approach, the aim is to mimic the dynamic variations and fluctuations in the levels of these interference substances that can commonly arise within the human body.

### 3.9 In Vitro Experiments

To evaluate the practical performance of the OECT-CGM system, in vitro experiments are conducted to simulate dynamic glucose variations in the human body. The testing setup comprised a glucose solution reservoir with both an inlet and outlet, a programmable syringe that allowed precise control of the injection speed, and a programmable injecting pump enabling us to modulate the glucose concentration in the reservoir (FIG. 13). During these experiments, two cycles of concentration change are performed within the range of 0 to 20 mM, and vice versa, with a gradual speed of 0.3 mM per minute. Each concentration change cycle is completed in approximately 2 hours, mimicking the potential fluctuations of glucose levels in the body.

### 3.10 In Vivo Tests

The in vivo test of the OECT-CGM for glucose tracking is performed using female SD rats (300 to 350 g). The rats are fasted for 20 hours but are provided with water to obtain a stable initial blood glucose level. First, for blood collection convenience, rats are anesthetized using 2.5% (v/v) isoflurane. To manipulate the rats' blood glucose levels, a 400 mM glucose solution is administered to the rats via a syringe at a dosage of 10 mL/kg, starting 40 minutes after the commencement of the experiment. The OECT-CGM are applied to the back of the rat and the sampling rate are set as 90 second/sample for 2 hours of monitoring. To validate the blood glucose level of the rats, the blood taken from the tail vein of rats is tested by a glucometer (Yuwell Accusure 590, China) at the frequency of 15 minutes for 2 hours.

It should be understood that the examples and embodiments described herein are for illustrative purposes only and that various modifications or changes in light thereof will be suggested to persons skilled in the art and are to be included within the spirit and purview of this application.

All patents, patent applications, provisional applications, and publications referred to or cited herein are incorporated by reference in their entirety, including all figures and tables, to the extent they are not inconsistent with the explicit teachings of this specification.

It should be understood that numerous specific details, relationships, and methods are set forth to provide a full understanding of the invention. One having ordinary skill in the relevant art, however, will readily recognize that the invention can be practiced without one or more of the specific details or practiced with other methods, protocols, reagents, cell lines and animals. The present invention is not limited by the illustrated ordering of acts or events, as some acts may occur in different orders and/or concurrently with other acts or events. Furthermore, not all illustrated acts, steps or events are required to implement a methodology in accordance with the present invention. Many of the techniques and procedures described, or referenced herein, are well understood and commonly employed using conventional methodology by those skilled in the art.

Unless otherwise defined, all terms of art, notations and other scientific terms or terminology used herein are intended to have the meanings commonly understood by those of skill in the art to which this invention pertains. In some cases, terms with commonly understood meanings are defined herein for clarity and/or for ready reference, and the inclusion of such definitions herein should not necessarily be construed to represent a substantial difference over what is generally understood in the art. It will be further understood that terms, such as those defined in commonly used dictionaries, should be interpreted as having a meaning that is consistent with their meaning in the context of the relevant art and/or as otherwise defined herein.

As used herein, the terms “approximately”, “substantially”, “substantially” and “about” are used to describe and explain small changes. When used in conjunction with an event or situation, the term can refer to situations where the event or situation occurs precisely, as well as situations where the event or situation occurs approximately. As used herein, with respect to a given value or range, the term “approximately” generally refers to a range of ±10%, ±5%, ±1% or ±0.5% of a given value or range. The range may be indicated in this disclosure as from one endpoint to another endpoint or between two endpoints. Unless otherwise stated, all ranges disclosed in this disclosure include endpoints. When referring to “substantially” the same numerical value or characteristic, the term may refer to values within ±10%, ±5%, ±1% or ±0.5% of the average value.

1. An organic electrochemical transistor-based continuous glucose monitoring system with minimal intrusiveness and no blood contact, comprising:
a hollow microneedle patch;
an adhesive and stretchable enzymatic hydrogel sensing membrane;
an organic electrochemical transistor-based glucose sensor;
a miniaturized readout system; and
a 3D printed resin encapsulation case coated with evaporated metal;
wherein the organic electrochemical transistor is capable of current regeneration, sensitivity adjustment and self-calibration.

2. The organic electrochemical transistor-based continuous glucose monitoring system of claim 1, wherein the hydrogel sensing membrane comprises:
an interpenetrating polymer network hydrogel comprising a sodium alginate first network and a polyacrylamide second network;
a semipermeable bioadhesive elastomer; and
glucose oxidase.

3. The organic electrochemical transistor-based continuous glucose monitoring system of claim 1, wherein the evaporated metal coating the 3D printed resin encapsulation case is selected from gold or platinum.

4. The organic electrochemical transistor-based continuous glucose monitoring system of claim 1, wherein the system is capable of tracking cell glucose metabolism and clinical blood glucose concentrations within a concentration range of 10⁻⁶ M to 10⁻¹ M.

5. The organic electrochemical transistor-based continuous glucose monitoring system of claim 1, wherein the system has a dimension of less than 2 cm×2 cm×0.5 cm.

6. The organic electrochemical transistor-based continuous glucose monitoring system of claim 1, wherein the signal-to-noise ratio is at least 50 dB.

7. The organic electrochemical transistor-based continuous glucose monitoring system of claim 1, wherein the current regeneration is performed by adjusting the gate voltage of the organic electrochemical transistor.

8. The organic electrochemical transistor-based continuous glucose monitoring system of claim 1, wherein the sensitivity adjustment comprises controlling the transconductance to adjust the anti-noise ability; and adjusting the gate voltage to adjust the linear range of detection.

9. The organic electrochemical transistor-based continuous glucose monitoring system of claim 1, wherein the self-calibration comprises normalizing the transconductance curves.
